(19) United States
(12) Patent Application Publication
(10) Pub. No.: US 2007/0078091 A1
(43) Pub. Date: Apr. 5, 2007

(54) Pharmaceutical combinations for compensating for a testosterone deficiency in men while simultaneously protecting the prostate

(76) Inventors: Doris Hubler, Schmieden (DE); Michael Oettel, Jena (DE); Lothar Sobek, Jena (DE); Walter Elger, Berlin (DE); Abdul-Abbas Al-Mudhaffar, Jena (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/517,301

(22) Filed: Sep. 8, 2006

Related U.S. Application Data
(63) Continuation of application No. 09/719,221, filed on Feb. 16, 2001.

(30) Foreign Application Priority Data
Sep. 6, 1998 (DE) 198 25 591.8

(51) Int. Cl. A61K 38/09; A61K 31/57; A61K 31/56; A61K 31/519; A61K 31/517; A61K 31/4709; A61K 31/138

(52) U.S. Cl. 514/15; 514/171; 514/170; 514/651; 514/252.16; 514/252.17; 514/308

(57) ABSTRACT

This invention relates to pharmaceutical combinations for compensating for an absolute and relative testosterone deficiency in men with simultaneous prophylaxis for the development of a benign prostatic hyperplasia (BPH) or prostate cancer. The combinations according to the invention contain a natural or synthetic androgen in combination with a gestagen, an antigestagen, an antiestrogen, a GnRH analog, a testosterone-5α-reductase inhibitor, an α-andreno-receptor blocker or a phosphodiesterase inhibitor. In comparison to the combinations according to the invention, any active ingredient by itself cannot achieve the desired goal.

The invention relates to pharmaceutical combinations for compensating for an absolute and relative testosterone deficiency in men with simultaneous prophylaxis for the development of a benign prostatic hyperplasia (BPH) or prostate cancer that contains a natural or synthetic androgen in combination with a gestagen, an antigestagen, an antiestrogen, a GnRH analog, a testosterone-5α-reductase inhibitor, an α-andreno-receptor blocker or a phosphodiesterase inhibitor.

Various endocrine functions vary during the course of the aging process.

The normal aging process in men is accompanied by a reduction in the testicular function, especially a reduction in the serum-testosterone level.

The serum-testosterone secretion is responsible for the secondary sex characteristics, libido and potency and also has an effect on the emotional and intellectual capabilities, on the erythropoiesis, bone metabolism, protein anabolism and muscle mass, fat distribution and certain CNS functions. In lowering the serum-testosterone level, a reduction of the libido and potency, as well as fatigue, reduction of the muscle mass, osteoporosis, hot flashes, profuse sweating and slight anemia can clinically occur.

An important role is ascribed to androgens for the development and manifestations of both benign prostatic hyperplasia (BPH) and prostate cancer, however.

At older ages, diseases of the prostate occur in clusters. In 50% of men over 50 years old, this leads to a non-malignant growth of the prostate (BPH).

Hypogonadal males or castrated males never develop a BPH. Geller, J.: Androgen Inhibition and BPH. in: Bhasin et al. (Editors): Pharmacology, Biology and Clinical Applications of Androgens. John Wiley, New York (1996).

In men with and without BPH, however, no differences in androgen concentrations in the serum exist [Lee, C., Prostate 6 Supple., 52-56 (1996), Levine, A. C. Trends Endocrinol. Metab. 6, 128-132 (1995); Serio, M. and Fiorelli, G. Mol. Cell. Endocrinol. 78, C77-C81 (1991), Cunningham, G. R.: Overview of Androgens on the Normal and Abnormal Prostate. In: Bhasin et al. (Editors). Pharmacology, Biology, and Clinical Applications of Androgens. John Wiley, New York (1996)], so that obviously the cellular metabolism of testosterone into 5α-dihydrotestosterone (DHT) and estradiol in the prostate, together with local growth factors, is of decisive importance for the development both of benign prostatic hyperplasia (BPH) and prostate cancer.

Both in men over 50 and in younger men with various chronic diseases and continuous stress, all indicated clinical symptoms in serum-testosterone levels demonstrably occur in clusters even at the lower standard limits of 12.0 to 15 nmol/l.

It is known from the literature or patent literature to treat androgen-dependent systemic diseases, such as, for example, the BHP and the prostate cancer, with antiandrogens by themselves—W94/26767 A1—or in combination with testolactone as an aromatase inhibitor—DE 3121152 A1—, with testosterone-5α-reductase inhibitor by itself—EP 0 547 691 A1; WO 95/13077 A1—or in combination with antiestrogens and/or aromatase inhibitors WO 91/00731 A1.

A testosterone replacement therapy without risk to the prostate is not indicated.

In the technical literature, it is shown that androgen substitution also improves physical and mental well-being as a person ages [Bagatell et al. J. Clin. Endocrin. Metab. 79: 561-567 (1994); Tenover, J. S. Endocrinology and Metabolism Clinics of North America 23: 878-892 (1994)].

The androgen substitution in older men with reduced serum-testosterone levels is still a controversial topic for a wide variety of reasons, however, and the increased risk of diseases of the prostate by overstimulation is always emphasized.

It is therefore inadvisable to undertake androgen replacement therapy in the older or prematurely aged man analogously to postmenopausal hormone substitution of the woman [Rolf, C. and E. Nieschlag: Seneszenz [Senescence] in E. Nieschlag and H. M. Behre (Editors): Andrologie—Grundlagen und Klinik der reproduktiven Gesundheit des Mannes [Andrology—Principles and Clinical Studies of the Reproductive Health of the Man]. Springer 1996: Jackson, J. A. et al. Arch. Intern. Med. 149: 2365-2366 (1989): Jockenhbvel, F. Androgensubstitution des älteren Mannes [Androgen Substitution of the Older Man]. In: Allolio and Schulte (Editors). Praktische Endokrinologie [Practical Endocrinology]. Urban & Schwarzenberg, Munich, pp. 416-419 (1996)].

Also, e.g., after an 8-month therapy of 23 men at the ages of 40-65 years with testosterone undecanoate (160 mg/day), Holmang, S. et al. Prostate 23, 99-106 (1996) could detect a 12% increase in size of the prostate.

In studies on male contraception with testosterone enanthate, an enlargement of the prostate was found in young men under exogenic testosterone administration by means of transrectal ultrasound studies [Wu, C. W. et al. Fertility and Sterility 65, 626-636 (1996); Wallace, E. M. et al. Int. J. Androl. 16: 35-40 (1993)].

Patent DE 196 10 645 A1 describes the use of dehydroepiandrosterone in combination with aromatase inhibitors for treatment of a relative and absolute androgen deficiency in men (hypoandrogenism). Aromatase inhibitors in terms of this patent are all those compounds that prevent the formation of estrogens from their metabolic precursors (here DHEA) by inhibiting the enzyme aromatase (inhibition of the biosynthesis).

Androgen therapy with simultaneous protection of the prostate is not indicated, however.

Patent WO 97/29735 claims androgens, antiandrogens, estrogens or antiestrogens containing transdermal systems, individually or in combination, for androgen therapy in the case of a deficiency of the testosterone level in hypogonadal men, for hormone substitution therapy in postmenopausal women and for hormonal contraception in men and in women.

Also, androgen therapy with simultaneous protection of the prostate is not indicated here.

The object of this invention is to define suitable combination preparations for compensating for an absolute and relative testosterone deficiency in men while simultaneously protecting the prostate and in this case to avoid the above-mentioned drawbacks and actions.

The object is achieved by the use according to the invention of combination preparations according to claim 1 for compensating for an absolute and relative testosterone deficiency with simultaneous therapy of the benign prostatic hyperplasia (BPH).

The use of the combination preparations according to the invention is preferably characterized in that natural androgen is one of the substances testosterone, testosterone undecanoate, dehydroepiandrosterone, dehydroepiandrosterone sulfate, testosterone propionate, testosterone enanthate, testosterone buciclate, testosterone cypionate or androstene dione, and the synthetic androgen is one of the substances 17-methyltestosterone, fluoxymesterone, danazol, mesterolone, nandrolone decanoate, nandrolone phenylpropionate, oxandrolone, oxymetholone, or stanazolol.

It has proven advantageous that the dosage of the androgen, for example of testosterone undecanoate, is 250 to 1500 mg i.m. every 4 to 14 weeks.

In this case, the administration of 1000 mg of testosterone undecanoate every 9 to 10 weeks is especially advantageous.

The use according to the invention of combination preparations is preferably characterized in that the gestagen component is one of the substances dienogest, levonorgestrel, gestodene, desogestrel, norgestimate, norethisterone, norethisterone acetate, levonorgestrel or progesterone, chloromadinone acetate, cyproterone acetate, medroxy progesterone acetate, megestrol acetate, dydrogesterone, trimegestone or nomegestrol.

In this case, it is advantageous that the dosage of the gestagen is 20 μg to 20 mg.

The antigestagen component is preferably

4-[17β-Hydroxy-17α-(methoxymethyl)-3oxoestra-4,9-dien-11β-yl]benzaldehyde-1(E)-oxime (J 912);

4-[-17β-methoxy-17α-(methoxymethyl)-3-oxo-estra-4,9-dien-11β-yl]-benzaldehyde-1(E)-{O-[(ethylthio)carbonyl)}-oxime (J 1042);

4-[9α,10α-epoxy-17β-hydroxy-17a-(methoxymethyl)-3-oxo-estr-4-en-11β-yl]-benzaldehyde-1(E)-oxime (J 1116);

4-[17β-methoxy-17α-(methoxymethyl)-3oxoestra-4,9-dien-11β-yl]benzaldehyde-1(E)-oxime (J 867);

4-[17β-hydroxy-17α-(methoxymethyl)-3oxoestra-4,9-dien-11β-yl)benzaldehyde-1(E)-{O-[(N-ethyl)-carbonyl)}-oxime (J 956);

11β-[(4-N,N-dimethylamino)-phenyl]-17β-hydroxy-17α-propinyl-estra-4,9-dien-3-one (RU 38 486—mifepristone);

11β-][(4-N,N-dimethylamino)-phenyl]-17α-hydroxy-17β-(3-hydroxypropyl-13α-methyl-gona-4,9-dien-3-one (ZK 98299—onapristone);

11β-(4-acetylphenyl)-17β-hydroxy-17α-propinyl-estra-4,9-dien-3-one (ZK 112993);

11β-[(4-N,N-dimethylamino)-phenyl]-17β-hydroxy-17α-(3-hydroxy-1-(Z)-propenyl)-estra-4,9-dien-3-one (ZK 98 734—lilopristone);

11β-[(4-N,N-dimethylamino)-phenyl]-17β-hydroxy-17α-(3-hydroxy-1-(Z)-propenyl)-estra-4-en-3-one (ZK 137 316);

11β-[(4-N,N-dimethylamino)-phenyl]-6B-methyl-4′,5′dihydrospiro-[estra-4,9-diene-17,2′(3′H)-furan]-3-one (ORG 31 710);

11β-[(4-N,N-dimethylamino)-phenyl]-73-methyl-4′,5′dihydrospiro-[estra-4,9-diene-17,2′(3′H)-furan]-3-one (ORG 31 806);

11β-(4-acetylphenyl)-(3′E)-ethylidene-4′,5′dihydrospiro-[estra-4,9-diene-17,2′(3′H)-furan]-3-one (ORG 33 628).

The antiestrogen component is preferably tamoxifen, raloxifene, panomifene, toremifene, iproxifene or idoxifene.

The GnRH-analog component is preferably buserelin, goserelin, nafarelin, triptorelin or deslorelin, leuprolide or leuprolide acetate.

The testosterone-5α-reductase-inhibitor component is preferably finasteride, epristeride, permixon, or turosteride.

The α-andreno-receptor-blocker component is preferably tolazoline, phentolamine, phenoxybenzamine, alfuzosin, or prazosin.

The phosphodiesterase-inhibitor component is preferably amrinone, milrinone, trapidil, papaverine, vesnarinone or sildenafil.

The object is achieved according to the invention by use of the combinations in different preparation or administration forms.

The pharmaceutical preparation forms can depict the combinations as a uniform form or else contain two separate formulations. In this case, they can be preparations for peroral use, e.g., tablets, capsules and coated tablets; percutaneous preparation forms, e.g., transdermal therapeutic systems (TTS) or gels, sprays or ointments; intranasal preparation forms, such as nasal spray or nose drops, rectal preparation forms such as suppositories and preparations for parenteral use, e.g., implants, pressed parts and ampoules.

The preparation forms are produced in a way that is known in the art with use of commonly used adjuvants and vehicles, as are described in, for example, “Remington's Pharmaceutical Sciences Handbook, Hack Pub. Co., N.Y., USA.”

A pharmaceutical combination for compensating for an absolute and relative testosterone deficiency in men with simultaneous prophylaxis of benign prostatic hyperplasia (BPH) was found.

In comparison to the combination according to the invention, any active ingredient by itself cannot achieve the desired goal to this extent and only with significant side effects.

With the combinations according to the invention, the DHT-stimulation that is caused by the administration of the androgens or overstimulation in the prostate is compensated for by the indicated components, such as gestagens, antigestagens, antiestrogens, GnRH-analogs, testosterone-5α-reductase inhibitors, α-andreno-receptor blockers or phosphodiesterase inhibitors.

In the example of the inhibition of the androgen-dependent cell proliferation in LNCaP prostate cells, the biological mechanism of action of the combination of 17β-hydroxy-17α-methyl-estra-4,9,11-trien-3-one (R 1881)+the 17α-cyano-methyl-17β-hydroxy-estra-4,9-dien-3-one (DIENOGEST=DNG) was examined.

To this end, the human prostate cancer cell LNCaP was cultivated under routine conditions in Dulbecco's modified Eagle medium (DMEM) with the addition of 10% FCS (fetal calf serum).

The cells were then cultured for 2 to 6 passages with DMEM and 10% DCC-FCS (steroid-depleted FCS), before it was used in a growth assay with 5% DCC-FCS.

For the test, the cells were saturated in 24-well plates (10,000 cells/well and ml).

After 24 hours, the steroids that were dissolved in ethanol were added to fresh test medium (final concentration of ethanol 0.1%), and the cells were incubated for 7 days at 37° (5% CO₂). The cell number was determined after this time in a cell counting and analysis system (CASY, Scharfe System GmbH).

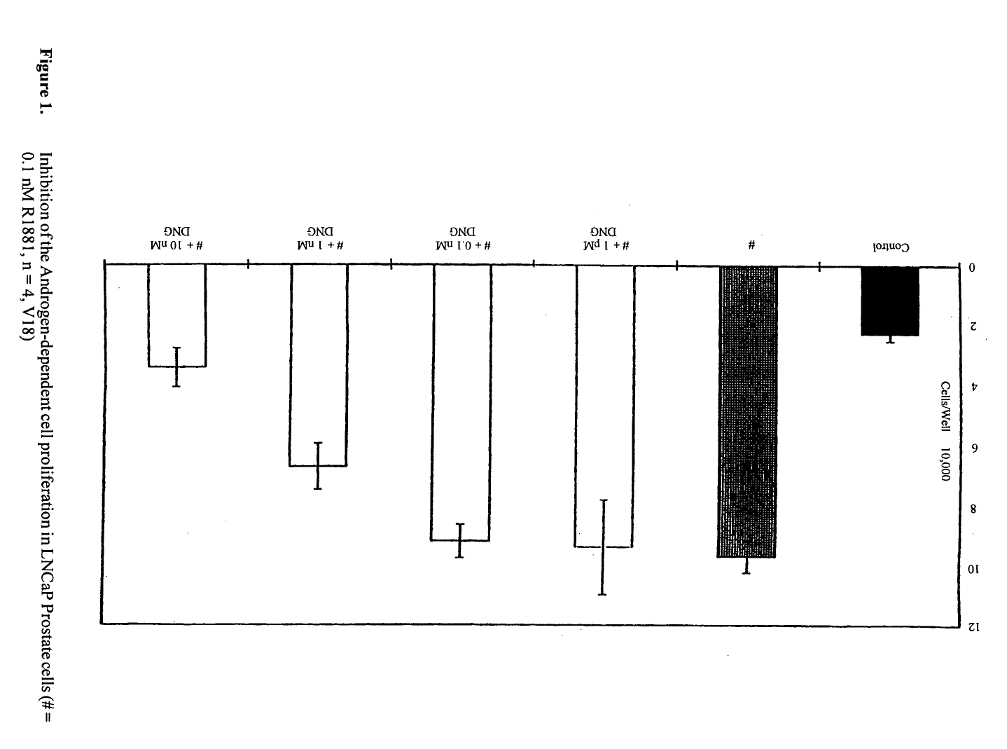

It can be seen from FIG. 1 that the very strong androgen 17β-hydroxy-17α-methyl-estra-4,9,11-trien-3-one (R1881) induces a cell growth. Based on the dose of the second component—here the gestagen DNG—however, the androgen-dependent cell proliferation of the prostate-cancer-cell line is inhibited. The dienogest action is first and foremost peripheral to the sex organs (Oettel, M. et al., Der Einfluβ einer Ethinylestradiol-Dienogest-Kombination auf die Serum-Androgen-Konzentrationen [The Effect of an Ethinylestradiol-Dienogest-Combination on the Serum-Androgen-Concentrations], Zentralblatt Gynäkol 119, 597-606, 1997).

The inhibition of a prostate growth that was induced by androgens and that was treated with a combination according to the invention, was also examined in the animal experiment.

To this end, 5 male NMRI mice of Møllegaard Breeding Centre Deutschland GmbH, Schönwalde weighing 28-30 g were castrated. Two weeks after the castration, testosterone propionate (TP) by itself—0.1 mg/animal—was administered to the control animals.

Also, two weeks after castration, the test animals were orally treated daily for one week with testosterone propionate (TP) 0.1 mg/animal/day s.c. and simultaneously with the following gestagens and the following dosages:

cyproterone acetate (CPA) at dosages of 0.1; 0.3; 1; 3 mg/animal/day dienogest (DNG) at dosages of 0.3; 1; 3; 10 mg/animal/day, chloromadinone acetate (CMA) at dosages 0.3; 1; 3; 10 mg/animal/day.

At the end of the test, the prostate weights of the mice were determined, and the test groups were compared.

In Table 1, the determined prostate weights of the treated mice are indicated during the course of the test.

Prostate Weights of Castrated Mice in mg (Mean Value ± S.D.)
after Combined Testosterone/Gestagen Treatment (n = 5
animals/group)

| | Gestagen dosage % groupings | | | | | |
|---|---|---|---|---|---|---|
| | | 0.1 | 0.3 | 1.0 | 3.0 | 10.0 |
| | | (mg/animal/ | (mg/animal/ | (mg/animal/ | (mg/animal/ | (mg/animal/ |
| | | day) | day) | day) | day) | day) |
| Intact | 3.0 ± 0.8 | | | | | |
| control | | | | | | |
| Castrated | 1.7 ± 0.7 | | | | | |
| control | | | | | | |
| TP | 4.4 ± 0.7 | | | | | |
| control | | | | | | |
| TP + CPA | | 2.7 ± 1.2* | 3.3 ± 1.3 | 2.2 ± 1.1* | 2.3 ± 0.8* | 2.7 ± 0.7* |
| TP + DNG | | | 2.7 ± 0.7* | 3.4 ± 0.5* | 3.1 ± 0.7* | 2.4 ± 0.9* |
| TP + CMA | | | 3.0 ± 1.0* | 3.3 ± 0.8 | 3.0 ± 1.1* | 2.5 ± 0.4* |

*significant p ≧ 0.05 (substance group vs. TP)

From Table 1, it can be seen that the selected pure testosterone dose—0.1 mg/animal/day—causes a clear increase of the prostate weight compared to the castrated and the intact control animals. The androgen/gestagen combination reduces the androgen-produced increase of the prostate weight—depending on the dosage of the gestagen—up to the range of prostate weights of intact comparison animals.

Thus, the compensating for the relative testosterone deficiency and the simultaneous protection of the prostate is demonstrably ensured.

With the combinations according to the invention, pharmaceutical agents are made available that compensate for a relative testosterone deficiency in men and simultaneously protect the prostate.

1-12. (canceled)

13. A method of compensating for an absolute or relative testosterone deficiency in a patient who has said testosterone deficiency with simultaneous prophylaxis in said patient for the development of a benign prostatic hyperplasia or a prostate cancer comprising administering to said patient a combination of an androgenically effective amount of a natural or synthetic androgen, and a gestagenically effective amount of a gestagen.

14. A method according to claim 13, wherein the natural androgen component is testosterone, testosterone undecanoate, dehydroepiandrosterone, dehydroepiandrosterone sulfate, testosterone propionate, testosterone enanthate, testosterone buciclate, testosterone cypionate or androstene dione.

15. A method according to claim 13, wherein the synthetic androgen component is 17-methyltestosterone, fluoxymesterone, danazol, mesterolone, nandrolone decanoate, nandrolone phenylpropionate, oxandrolone, oxymetholone, or stanazolol.

16. A method according to claim 13, wherein the gestagen component is dienogest, levonorgestrel, gestodene, desogestrel, norgestimate, norethisterone, a norethisterone ester, progesterone, chloromadinone acetate, cyproterone acetate, medroxy progesterone acetate, megestrol acetate, dydrogesterone, trimegestone or nomegestrol.

17. A method of claim 13, wherein said combination is used in the form of a tablet, capsule, coated tablet, transdermal therapy system, ampoule, suppository, gel, ointment, nose drop, implant, pressed part or biodegradable microspheres.

18. A method of claim 13, wherein the dosage of androgen is 250-1500 mg i.m. every 4 to 14 weeks and the dosage is 20 μg to 20 mg.

19. A method according to claim 13, wherein prophylaxis for the development of a prostate cancer is achieved.

20. A method according to claim 19, wherein the gestagen is dienogest.

21. A method according to claim 13, wherein the androgen component is 17β-hydroxy-17α-methyl-estra-4,9,11-trien-3-one.
